(19) United States
(12) Patent Application Publication
(10) Pub. No.: US 2022/0008677 A1
(43) Pub. Date: Jan. 13, 2022

(54) Humidity Nasal Prong Cannula Assembly

(71) Applicant: Swirl Technologies Pty Ltd., Mitcham, Victoria (AU)

(72) Inventor: Ian Bird, Mitcham, Victoria (AU)

(21) Appl. No.: 17/293,171

(22) PCT Filed: Nov. 11, 2019
(86) PCT No.: PCT/AU2019/051240

§ 371 Date: May 12, 2021

(30) Foreign Application Priority Data
Nov. 12, 2018 (AU) 2018904401

(51) Int. Cl. A61M 16/10; A61M 16/06

(52) U.S. Cl. CPC A61M 16/1045; A61M 16/107; A61M 16/0672; A61M 16/101

(57) ABSTRACT

A unitary humidity oxygen nasal prong cannula assembly includes two oxygen supply assemblies. Each assembly includes a body (5) having an oxygen inlet (7) and a prong (9) extending from the body (5). The prong (9) is configured for insertion into a nostril of a patient. The body and the prong define a passageway for supplying oxygen to the nostril of the patient. The assembly also includes a heat and moisture exchange (“HME”) device (13) for providing moisture and heat to cold oxygen supplied to the cannula from an oxygen concentrator. The HME device forms a part of the body and is configured so that cold oxygen passing through the passageway passes through the HME device.

### FIELD OF THE INVENTION

The present invention relates to a humidity oxygen nasal prong cannula assembly.

The present invention also relates to a portable oxygen concentrator assembly that includes a humidity oxygen nasal prong cannula assembly.

### BACKGROUND OF THE INVENTION

A problem with known oxygen concentrators that supply pure oxygen to patients is that the cold oxygen can cause nasal passages to dry out and this dryness and the coldness of the oxygen can be a source of discomfort for patients.

It is known to use heat moisture exchange (HME) devices with known oxygen concentrators as a means of supplying humidity and warmth to patients.

The use of oxygen concentrators and HME devices is a viable option for hospitals and other healthcare facilities where there is space for the equipment.

The invention is concerned with providing moisture and potentially some heat to cold oxygen produced by a portable oxygen concentrator that is located away from conventional HME devices that are used with oxygen concentrators at hospitals and other healthcare facilities. The objective is that the moisture and potentially some heat be experienced as humidity and warmth by patients receiving oxygen via the portable oxygen concentrator.

In particular, the invention is concerned with providing moisture and potentially some heat to oxygen produced by a portable oxygen concentrator to provide a higher level of comfort for patients than is currently available to patients receiving oxygen from the portable oxygen concentrator.

The term “oxygen concentrator” is understood herein to mean a device that filters air, compresses the filtered air, and removes nitrogen from the air, and supples “pure” oxygen (at least 90 vol. % oxygen).

Typically, a portable oxygen concentrator produces an equivalent of 1-5 litres per minute of continuous oxygen flow and operates with pulse flow or “demand flow” to deliver oxygen only when the patient is inhaling. Portable oxygen concentrators typically plug into an electrical outlet and may have an internal battery or external battery pack for operation away from home.

The term “heat and moisture exchange”, i.e. “HME”, device is understood herein to mean a device that removes and retains moisture from expired gas from a patient and transfers moisture and heat to oxygen that passes through the device to the patient during periods of inspiration by the patient. The mechanisms of humidification and heating via HME devices are summarised below.
HME devices contain a membrane that includes a layer of foam or paper embedded with a hydroscopic salt, such as calcium chloride.
Expired gas passing through the foam/paper layer is cooled as the gas crosses the membrane, resulting in condensation and release of the mass enthalpy of vaporisation to the HME layer in the membrane.
On inspiration of cold oxygen, absorbed heat in the membrane warms oxygen passing through the membrane and the hygroscopic salt releases water molecules, and the water is transported as moisture with the warmed oxygen to the patient.
Warming and humidification is thus regulated by the moisture content of the expired gas.

A “nasal prong cannula” is understood herein to be a device that is used to deliver supplemental oxygen to a person in need of respiratory help.

Known nasal prong cannulas comprise a lightweight tube which has two prongs that, in use, are placed in the nostrils of a person and through which, in use, oxygen flows into the nostrils and then the lungs of the person.

The above description of the background of the invention is not to be taken as an admission of the common general knowledge in Australia or elsewhere.

### SUMMARY OF THE INVENTION

The invention is based on a realisation that an effective option for providing moisture and potentially some heat to cold oxygen to be supplied to a patient via a portable oxygen concentrator is to combine together into one assembly a heat moisture exchange (“HME”) device and a nasal prong cannula for supplying oxygen to the patient.

More particularly, the invention is based on a realisation that cold oxygen from a portable oxygen concentrator can be provided with moisture by incorporating a HME device into a nasal prong cannula and configuring the resultant cannula assembly so that expired air from a patient passes through the filter of the HME device in the assembly and provides moisture to the filter material that can be transferred to oxygen that is subsequently supplied to the patient and is then experienced as humidity by the patient.

In broad terms, the invention provides a humidity oxygen nasal prong cannula assembly for a patient that includes two oxygen supply assemblies, each assembly including:
(a) a body and a prong extending from the body, the body including an oxygen inlet, and the prong being configured for insertion into a nostril of a patient and having an oxygen outlet, the body and the prong defining a passageway for supplying oxygen to the nostril of the patient; and
(b) a heat and moisture exchange (“HME”) device for providing moisture to cold oxygen supplied to the cannula from an oxygen concentrator, such as a portable oxygen concentrator, that, in use, is experienced as humidity by the patient, with the HME device forming a part of the body and being configured so that cold oxygen passing through the passageway passes through the HME device.

The humidity oxygen nasal prong cannula of the invention is not confined to use with portable oxygen concentrators and is suitable to be used with other oxygen concentrators.

It is noted that the invention has particular application for use with portable oxygen concentrators.

It is also noted that the invention has particular application for use with babies.

Each HME device may be configured to provide an additional 10%, typically at least 15%, more typically at least 20%, and more typically at least 25% moisture, to cold oxygen passing through the oxygen supply assemblies.

The humidity oxygen nasal prong cannula assembly may be configured so that expired air from the patient passes through the HME devices and provides moisture to the devices.

The humidity oxygen nasal prong cannula may be configured to allow additional moisture to be added to the HME devices to adjust the moisture and heat in oxygen supplied to the patient via the cannula assembly.

The HME devices may be any suitable devices.

The invention also provides a portable oxygen concentrator assembly that includes:
(a) a portable oxygen concentrator;
(b) the above-described humidity oxygen nasal prong cannula assembly; and
(c) tubing interconnecting the portable oxygen concentrator and the humidity oxygen cannula.

### BRIEF DESCRIPTION OF THE DRAWINGS

The invention is described further by way of example only with reference to the accompanying drawings, of which:

FIG. 1 is perspective view of one, but not the only, embodiment of a humidity oxygen nasal prong cannula assembly in accordance with the invention;

FIG. 2 is a side view of the humidity oxygen nasal prong cannula assembly shown in FIG. 1 in the direction of the arrow A in the Figure;

FIG. 3 is an end view of the humidity oxygen nasal prong cannula assembly shown in FIG. 1 from one end of the cannula;

FIG. 4 is an end view of the humidity oxygen nasal prong cannula assembly shown in FIG. 1 from the other end of the cannula;

FIG. 5 is a top view of the humidity oxygen nasal prong cannula assembly shown in FIG. 1; and

FIG. 6 is a diagram illustrating one embodiment of a portable oxygen concentrator assembly in accordance with the invention.

### DETAILED DESCRIPTION OF A SPECIFIC EMBODIMENT

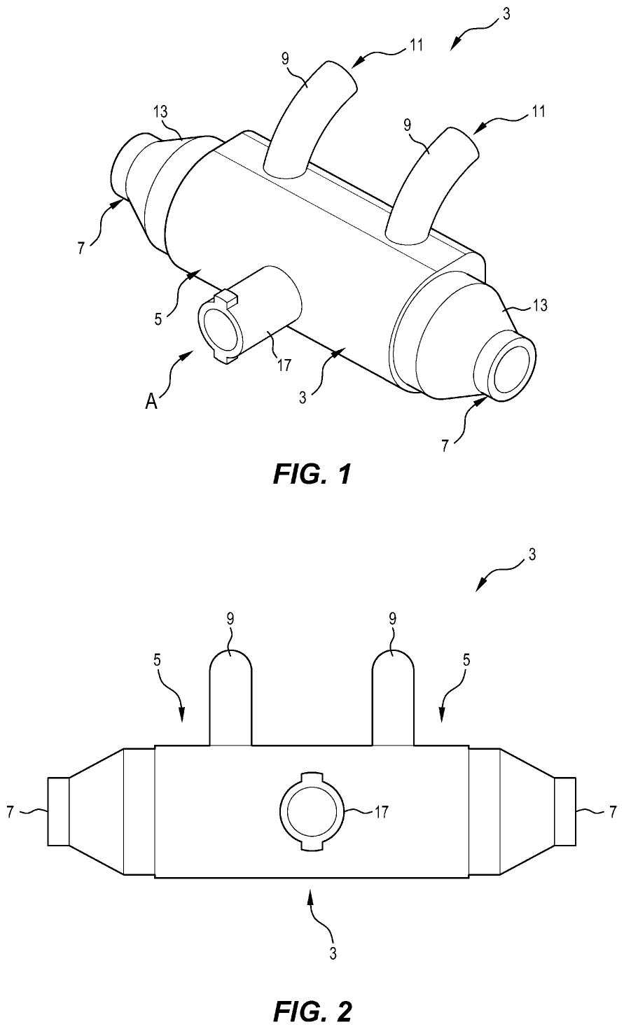

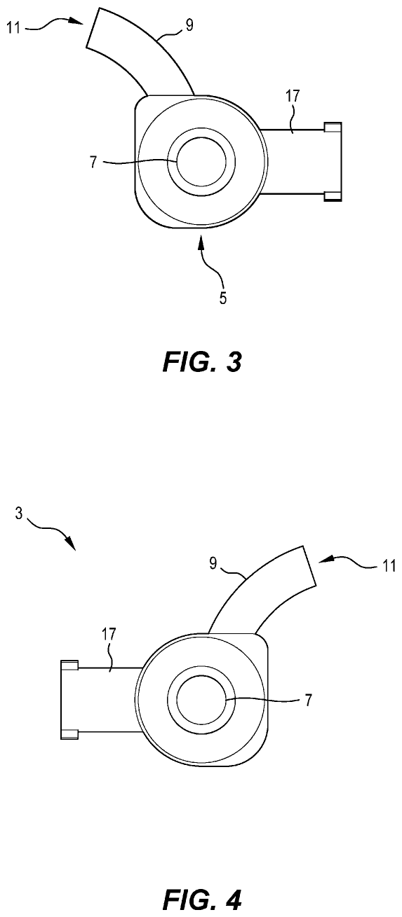

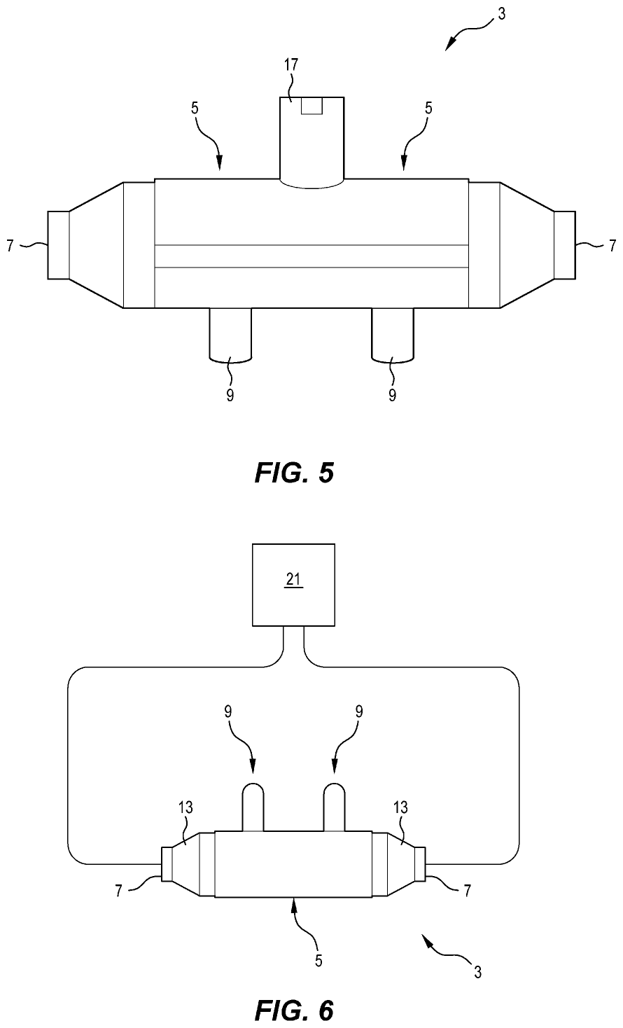

The embodiment of the humidity oxygen nasal prong cannula assembly of the invention shown in FIGS. 1-5 is one of a number of possible embodiments of the invention.

The humidity oxygen nasal prong cannula assembly 3 shown in the Figures includes two oxygen/air supply assemblies, each assembly including:
(a) a body 5 and a prong 9 extending from the body 5, the body including an oxygen inlet 7, and the prong 9 being configured for insertion into a nostril of a person and having an oxygen outlet 11, the body 5 and the prong 9 defining a passageway for supplying oxygen to the nostril of the person, and
(b) a HME device 13 for providing moisture and potentially some heat to cold oxygen supplied to the cannula assembly 3 from an oxygen concentrator (not shown), such as a portable oxygen concentrator 21 (see FIG. 6), with the moisture being experienced as humidity by the patient, with the HME device forming a part of the body 5 and being configured so that cold oxygen passing through the passageway passes through the HME device.

The Figures show that the two oxygen/air supply assemblies are formed as a unitary humidity oxygen nasal prong cannula assembly 3.

With regard to FIG. 1, the unitary humidity oxygen nasal prong cannula assembly 3 includes (a) a central generally cylindrical hollow barrel section having an internal wall or other suitable barrier (not shown) that separates the hollow barrel section into two oxygen/air supply assemblies, (b) the prongs 9 extending from the hollow barrel section, and (c) the HME devices 13 at opposite ends of the hollow barrel section. The HME devices 13 are shown as being tapered from an inner end to an outer end and transition the wider diameter hollow barrel section to the narrower diameter inlets 7. It can be appreciated that, in use, oxygen flowing into the inlets 7 flows through the passageways (not shown) defined by the HME devices 13 and the hollow barrel section and through the prongs 9 to the patient.

The HME devices 3 may be any suitable devices that include filter material for removing and retaining moisture from expired gas from a patient and transferring humidity to oxygen that passes through the devices to the patient during periods of inspiration by the patient.

The humidity oxygen nasal prong cannula assembly 3 also includes a luer lock 17 that can be unscrewed to allow additional moisture to be supplied to the filter material of the HME devices 13. The additional moisture may be provided from any suitable source. The assembly 3 may be configured to supply additional moisture continuously or periodically on a predetermined basis, or in response to moisture in oxygen passing through the assembly 3 falling below a threshold level or on any other suitable basis.

The humidity oxygen nasal prong cannula assembly 3 may be made from any suitable materials.

Typically, the adjustable prong nasal cannula assembly 3 is made from plastics materials.

The embodiment of the portable oxygen concentrator assembly generally identified by the numeral 19 in FIG. 6 includes the above-described humidity oxygen nasal prong cannula assembly 3, a portable oxygen concentrator 21, and two separate lengths of tubing connecting the to the inlets 7 of the humidity nasal prong cannula assembly 3.

In use, it is a straightforward exercise to connect together the assembly shown in FIG. 6, with the humidity oxygen nasal prong cannula assembly 3 positioned on a patient. Cold oxygen produced by the portable oxygen concentrator 21 is supplied to the patient via the tubing 23 and the humidity oxygen nasal prong cannula assembly 3. Oxygen flows from the portable oxygen concentrator 21 through the humidity oxygen nasal prong cannula assembly 3 to the patient and exhaled air from the patient flows through the humidity oxygen nasal prong cannula assembly 3. The process of exhalation causes release of moisture to the HME devices 13. Oxygen flowing through the HME devices 13 in the humidity oxygen nasal prong cannula assembly 3 to the patient picks up moisture from the HME devices 13 and this is transferred to the patient and experienced by the patient as humidity. The moisture released from expired air to the HME devices 13 returns moisture to the HME devices 13. Each HME device 13 is configured to provide an additional 10%, typically at least 15%, more typically at least 20%, and more typically at least 25% moisture, to cold oxygen passing through the oxygen supply assemblies

It can readily be appreciated that the humidity oxygen nasal prong cannula assembly 3 is a compact and lightweight unit that is well-suited for short-term use by patients, particularly in situations whether the patients are being supplied with oxygen for portable oxygen concentrators. Particularly in these situations, the humidity oxygen nasal prong cannula assembly 3 makes it possible to supply moisture and potentially some heat to oxygen produced by a portable oxygen concentrator to provide a higher level of comfort for patients than is currently available to patients receiving oxygen from the portable oxygen concentrator. In particular, the humidity oxygen nasal prong cannula assembly 3 makes it possible for patients in hospitals or other healthcare facilities to leave these facilities for short time periods, say 2-3 house, and make short recreational outings without substantial difficulty and be supplied with oxygen having humidity while on the trips.

Many modifications may be made to the embodiment of the invention described above without departing from the spirit and scope of the invention.

1. A humidity oxygen nasal prong cannula assembly that includes two oxygen supply assemblies, each assembly including:
(a) a body and a prong extending from the body, the body including an oxygen inlet, and the prong being configured for insertion into a nostril of a patient and having an oxygen outlet, the body and the prong defining a passageway for supplying oxygen to the nostril of the patient, and
(b) a heat and moisture exchange (“HME”) device for providing moisture to cold oxygen supplied to the cannula from an oxygen concentrator that, in use, is experienced as humidity by the patient, with the HME device forming a part of the body and being configured so that cold oxygen passing through the passageway passes through the HME device.

2. The humidity oxygen nasal prong cannula assembly defined in claim 1 configured so that expired air from the patient passes through the HME devices and provides moisture to the devices.

3. The humidity oxygen nasal prong cannula assembly defined in claim 2 configured to allow additional moisture to be added to the HME devices.

4. A portable oxygen concentrator assembly that includes:
(a) a portable oxygen concentrator assembly;
(b) the humidity oxygen nasal prong cannula defined in claim 1; and
(c) tubing interconnecting the portable oxygen concentrator and the humidity oxygen nasal prong cannula.

5. The humidity oxygen nasal prong cannula assembly defined in claim 2, wherein each HME device is configured to provide an additional 10% moisture to cold oxygen supplied to the cannula from the oxygen concentrator.

6. The humidity oxygen nasal prong cannula assembly defined in claim 1, further comprising (a) a central hollow barrel section having an internal wall that separates the hollow barrel section into two oxygen supply assemblies, (b) the prongs of the oxygen supply assemblies extending from the hollow barrel section, and (c) the HME devices of the oxygen supply assemblies at opposite ends of the hollow barrel section.

7. The humidity oxygen nasal prong cannula assembly defined in claim 6, wherein the hollow barrel section includes a luer lock that can be unscrewed to allow additional moisture to be supplied to the HME devices.

8. The humidity oxygen nasal prong cannula assembly defined in claim 6, wherein the HME devices taper from an inner end connected to the hollow barrel section to an outer end and transition from hollow barrel section to the oxygen inlet, with the hollow barrel section having a wider diameter than the oxygen inlet.

9. The humidity oxygen nasal prong cannula assembly defined in claim 1, wherein the HME devices include filter material for removing and retaining moisture from expired gas from the patient and transferring humidity to oxygen that passes through the devices to the patient during periods of inspiration by the patient.
